(19) United States
(12) Patent Application Publication
(10) Pub. No.: US 2013/0337453 A1
(43) Pub. Date: Dec. 19, 2013

(54) EXTRACELLULAR MITOCHONDRIA-BASED SCREENING AND TREATMENT

(71) Applicants: Theoharis C. Theoharides, Brookline, MA (US); Bodi Zhang, Boston, MA (US)

(72) Inventors: Theoharis C. Theoharides, Brookline, MA (US); Bodi Zhang, Boston, MA (US)

(73) Assignee: TUFTS UNIVERSITY, BOSTON, MA (US)

(21) Appl. No.: 13/880,736

(22) PCT Filed: Oct. 21, 2011
(86) PCT No.: PCT/US11/57405

§ 371 Date: Sep. 9, 2013

Related U.S. Application Data
(60) Provisional application No. 61/405,414, filed on Oct. 21, 2010.

(51) Int. Cl. C12Q 1/68

(52) U.S. Cl. CPC C12Q 1/6883
USPC 435/6.12; 436/501; 435/7.92

(57) ABSTRACT

Based on novel findings that mitochondrial dynamics regulate mast cell secretion of pre-stored TNF stimulated by SP, novel methods and compositions related to extracellular mitochondrial DNA (mtDNA) are provided for the diagnosis and treatment of diseases brought on by malfunctioning immune activities, e.g., inflammatory, autoimmune diseases, and neurodegenerative diseases such as ASD.

### CROSS-REFERENCE TO RELATED APPLICATIONS

This application claims priority to and the benefit of U.S. Patent Application Ser. No. 61/405,414, filed on Oct. 21, 2010, the entire contents of which are incorporated herein by reference.

### STATEMENT RE FEDERALLY FUNDED RESEARCH OR DEVELOPMENT

This invention was made with Government support under Grant No. R01 AR47652, awarded by the National Institutes of Health. The government has certain rights in this invention.

### BACKGROUND OF THE INVENTION

Inflammation is a complex response of the body to rid harmful stimuli and to heal itself, such as to remove a thorne and heal a wound. However, prolonged inflammation can lead to many diseases that can damage the tissue and cause pain and disabilities, such as rheumatoid arthritis (RA) and coronary atherosclerosis. Some such inflammatory diseases are also characterized as autoimmune diseases, such as psoriasis and systemic lupus erythematosus (SLE). Neurodegenerative diseases, such as Alzimhermer's and autistic spectrum disorder (ASD) may also have an inflammatory basis. Pathological inflammation and auto-immunity may overlap in some diseases.

The pathophysiology of many inflammatory diseases involves Tumor Necrosis Factor (TNF). Mast cell is the only cell type that stores pre-formed TNF in secretory granules (Olszewski, et al. 2007, J. Immunol. 178:5701-09), and constitutes a major source of rapid (1-30 minutes) TNF secretion (Gibbs et al., 2001, Exp Dermatol 10:312-20). Mast cells are bone marrow-derived immune cells that also secrete other pre-stored mediators such as histamine and tryptase through degranulation, as well as newly synthesized cytokines including interleukin-4 (IL-4) and interleukin-6 (IL-6), in response to allergic or neuropeptide triggers (Theoharides, et al. 2006, Ann NY Acad Sci 1088:78-99). Therefore, to better understand the inflammatory process and the various disorders brought about by prolonged inflammation, more needs to be learned about the processes during which mast-cell secretion of pre-stored TNF is triggered and carried out.

Mast cell secretion, especially degranulation during which vesicles called granules release cytotoxic substances from the cell as part of an immune response, is known to require intracellular calcium and energy production. Mitochondria are the primary energy-generating organelles in eukaryotic cells that participate in multiple intracellular processes, including calcium buffering (Wallace, 2005, Annu. Rev Genet. 39:359-07), many of which require mitochondrial translocation (Youle, et al. 2005, Nat. Rev Mol. Cell Biol. 6:657-63). These unique abilities enable mitochondria to participate in various cell behaviors, such as neuronal development, cell migration, and insulin secretion. Such functions require mitochondrial fission regulated by the cytoplasmic protein Drp1. Recent findings further indicate that Drp1 is regulated by calcineurin (Youle et al. 2005). However, there is a knowledge gap in mitochondrial dynamics as it pertains to mast cell secretion, and how this relates to inflammatory diseases, such as atopic dermatitis (AD) (Kawakami, et al. 2009, Curr. Opin. Immunol. 21:666-78), as well as other diseases of an autoimmune nature.

### SUMMARY OF THE INVENTION

The present invention is based on several novel discoveries and new data. First, we found that human mast cell degranulation leads to extracellular release of mitochondrial components including mitochondrial DNA (mtDNA) and ATP without causing cell death. Second, we found that extracellular mitochondrial components can stimulate mast cell degranulation and generate immune actions in a variety of tissues that could lead to effects including inflammation. Specifically, isolated mitochondrial components stimulated degranulation and de novo synthesis of IL-8 and VEGF in mast cells, IL-8 and VEGF production in cultured human keratinocytes, as well as VEGF and TNF production from primary human microvascular endothelial cells. Finally, mtDNA was detected in the serum of patients with severe psoriasis, proving the clinical feasibility of diagnosing autoimmune skin diseases and other inflammatory diseases with the presence of extracellular mtDNA.

Extracellular presence of mtDNA is normally absent except for cell death. Accordingly, a first aspect of the present invention relates to diagnosing various diseases through the detection of extracellular mitochondrial components associated with a disease process in a patient while confirm the lack of cell death. In one embodiment, a method is provided by the present invention for diagnosing an autoimmune disease, an inflammatory disease, and/or a neurodegenerative disease. The method includes the steps of detecting the presence of at least one extracellular mitochondrial component in a biological sample obtained from a patient as a mitochondria-specific marker or biomarker indicative of said disease, and confirming the absence of an indicia of cell apoptosis or necrosis in the same biological sample. In some cases, the presence of such component, especially when above a preselected threshold or at an elevated level, serves to indicate abnormal immune activities underlying the disease. The sample can be selected from the group consisting of plasma, serum, urine, lymph, cerebrospinal fluid, colonic fluid, nasal fluid, vaginal secretion, skin biopsy and other tissue biopsy.

Various diseases can be diagnosed using methods and devices provided by the present invention. These diseases include autoimmune diseases such as Churg-Strauss Syndrome, Coeliac disease, Hashimoto's thyroiditis, Goodpasture Syndrome, Graves' disease, inflammatory bowel disease, psoriasis, psoriatic arthritis, rheumatoid arthritis (RA), S\ddot{j}ogren's syndrome and systemic lupus erythematosus (SLE). Chronic inflammatory diseases that can be diagnosed using methods of the present invention include allergy, amyotrophic lateral sclerosis (ALS), asthma, chronic inflammatory disorder, atopic dermatitis, coronary atherosclerosis, interstitial cystitis, diabetes mellitus type 1 (IDDM), idiopathic thrombocytopenic purpura, multiple sclerosis and chronic pancreatitis. Further examples of diagnosable diseases include neurodegenerative diseases such as autistic spectrum disorder (ASD) (e.g., autism), chronic fatigue syndrome, chronic prostatitis, fibromyalgia, vitiligo and Parkinson's Disease. Other diseases that can be diagnosed by methods of the invention include mastocytosis, and seronegative RA.

In one feature, an embodiment according to the present invention includes the use of a second marker or biomarker for the suspected disease. Such second biomarker can be a known biomarker associated with the underlying disease. The diagnosis may be reached after viewing results from the two biomarkers separately or in combination. Use of multiple biomarkers may enhance the specificity of the test by reducing the chance of a false positive. In one embodiment, the second biomarker is an antinuclear antibody (ANA). In another embodiment, the second biomarker is neurotensin, especially for diagnosing autistic disorders (Angelidou et al. J of Neuroinflammation, 2010, 7:48).

In various embodiments, the (first) mitochondria-specific marker used in the present invention can be one or more of the following mitochondrial components: peptidoglycan, DNA, formyl-peptides, cytochrome C, and ATP. In a particular embodiment, the marker includes a unique region of mtDNA in the sample. In one example, the marker is one or more CpG dinucleotides in the mitochondrial DNA.

To distinguish the case where mitochondrial components are released extracellularly due to inflammation from the case where their presence is due to cellular necrosis or apoptosis, in one embodiment, the method of the present invention further seeks to detect damage-associated molecular patterns (DAMPs) released after cell apoptosis or necrosis. The presence of DAMPs would indicate conditions other than chronic inflammation at issue, such as trauma or cancer. In another embodiment, trypan blue exclusion method is used to confirm cell viability in the sample.

In a further embodiment of the present invention, a diagnostic kit is provided for diagnosing an autoimmune, inflammatory and/or neurodegenerative disease. The kit includes reagents for detecting, in a biological sample obtained from a patient, the presence of at least one extracellular mitochondrial component as a marker indicative of the disease. For example, the kit may include reagents for conducting PCR or quantitative PCR (qPCR) such as a pair of primers comprising sequences configured to amplify at least a region of a mitochondrial DNA. The target region, in one embodiment, comprises one or more CpG dinucleotide islands. The kit, in various embodiments, may include reagents for detecting the presence of other extracellular mitochondrial components or an indication of such presence (e.g., through detection and/or measurement of an antibody against such a component), as well as a second marker that is not related to mitochondria for the suspected disease. Further, the kit of the invention can include reagents for confirming the lack of cell death including apoptosis and necrosis.

A second aspect of the present invention relates to treatment of various autoimmune, chronic inflammatory and/or neurodegenerative diseases, including those described hereinabove, through inhibition of extracellularly released mitochondrial components. In one embodiment of the invention according to this aspect, a pharmaceutical composition is provided with a therapeutically effective amount of an agent capable of inhibiting at least one extracellular mitochondrial component activity, and a pharmaceutically acceptable excipient, carrier or diluent. For example, the agent can be an inhibitor of one or more of the following: extracellular mtDNA, extracellular mitochondrial peptidoglycan, ATP, a cellular receptor activated by mtDNA. In a particular embodiment, the agent comprises a DNA sequence substantially complementary to and capable of neutralizing extracellular mtDNA.

Besides neutralizing or inhibiting the activity of extracellular mitochondrial components, treatment goals can also be achieved through inhibiting one or more steps that lead to the release of mitochondrial components and the release itself. In one embodiment of the invention, a pharmaceutical composition is provided that includes a therapeutically effective amount of an agent capable of inhibiting at least one cellular function selected from: calcium influx, calcineurin activation, Drp1 activation, mitochondrial fission, mitochondrial translocation, and extracellular mitochondrial component release. The pharmaceutical composition may further include a pharmaceutically acceptable excipient, carrier or diluent. The agent may be selected from the group consisting of a flavonoid, and a third generation histamine-1 receptor antagonist. The flavonoid may be selected from the group consisting of apigenin, curcumin, epigallocatechin, luteolin, naringin, and quecetin. The flavonoid may be luteolin in a liposomal formulation. In one embodiment, the histamine-1 receptor antagonist is rupatadine.

In one feature, the present invention provides a method for treating a disease by administering to a patient a therapeutically effective amount of any of the pharmaceutical composition embodiments described herein. The method can further include administering to the patient a second medication such as a known treatment for the disease as a combined regimen. The disease can be an autoimmune, inflammatory, or neurodegenerative disease. In some embodiments, the pharmaceutical composition of the invention is administrated orally, parentally or topically.

It should be understood that this application is not limited to embodiments disclosed in the Specification. Instead, the application is intended to cover modifications and variations that are within the scope of those of sufficient skill in the field, and as defined by the claims. The foregoing and other objects, aspects, features and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1 illustrates that peptide Substance P (SP) stimulated pre-stored TNF secretion is associated with mitochondrial fission in human mast cells. LAD2 cells, mast cells derived from a human leukemia patient,were stained with MitoTracker probe (20 nM) for 20 min (middle panels) and LysoTracker DND probe (50 nM) for 30 min (left panels). The cells were then moved to glass bottom culture dishes and observed by TSC SP2 Confocal microscopy. FIG. 1A shows mitochondrial distribution of resting (upper panels) and degranulating (bottom panels) mast cells stimulated with SP (2 μM) for 30 min in comparison to the control. The left panels show granules in green. The middle panels represent mitochondria florescence. The right panels represent images merged from the two previous panels. FIG. 1B shows the percentage of mast cells with translocated mitochondria in resting and degranulating cells stimulated by SP (2 μM) for 30 min. Merged images (as described above) of 100 cells randomly selected from each experiment (n=3) are scored by three different operators each time. FIG. 1C shows the number of mitochondria per cell within 1 μm to the cell surface. Confocal images from 19 resting cells and 20 degranulating cells (SP 2 μM, 30 min) were randomly selected for analysis. Mitochondria numbers were calculated by ImageJ software using a “mitochondria calculator” plugin.

FIG. 2 illustrates that SP-stimulated degranulation and pre-stored TNF secretion is blocked by the mitochondrial fission protein Drp1 inhibitor (MDIVI-1) and Drp1-silencing siRNA. LAD2 cells were treated with MDIVI-1 (40 μM) for 15 min at 37° C. FIG. 2A: Mitochondrial morphology was evaluated by Confocal microscopy in normal LAD2 cells with or without stimulation with SP (2 μM, 30 min, 37° C.). Left panels represent granules; middle panels show mitochondria and the right panels are merged from the previous two. The images shown are representative of more than 40 cells studied. FIG. 2B shows β-hexosaminidase (beta-hex) and FIG. 2C pre-stored TNF secretion. LAD2 cells were pretreated with MDIVI-1 (40 μM) for 30 min and then stimulated with SP (1 or 2 μM) for 30 min at 37° C. β-hexosaminidase was measured spectrophotometrically and TNF was measured by ELISA. FIG. 2D shows β-hexosaminidase release stimulated by SP (2 μM, 30 min, 37° C.) in LAD2 cells treated with Drp1 siRNA. β-hexosaminidase release was used as an index of mast cell degranulation.

FIG. 3 is a Western blot image of Drp1 phosphorylation at amino acid Ser-616 after SP (2 μM) stimulation either with or without extracellular calcium. Antibodies against the Ser-616 phosphorylated form of Drp1 was purchased from Cell Signaling based in Massachusetts, USA.

FIG. 4 illustrates Drp1 and calcineurin gene expression in skin from AD patients as compared to healthy controls. Expression of Drp1 and calcineurin mRNA level were measured by quantitative RT-PCR and were normalized to GAPDH. Skin expression, i.e., mRNA level, of (A) Drp1 (controls n=10; patients n=10), (B) calcineurin (controls n=10; patients n=9) TaqMan was performed with cDNA reverse transcribed from 300 ng RNA from each sample (*p<0.05).

FIG. 5A consists of electron photomicrographs showing mitochondrial translocation to the cell surface in human mast cells where the left side picture shows normal and the right side shows SP-activated human skin mast cells, respectively. Mitochondria are shown within white rectangles. White asterisks represent released granular materials. FIG. 5B charts the number of mitochondria visible under electron microscope and FIG. 5C charts the percentage of mitochondria close to the cell surface in each cell as semi-quantitated from high magnification glossy prints by three independent operators. Magnification: 13,800×; (n=3; *p<0.05 compared to control).

FIG. 6 illustrates human LAD2 mast cells release mitochondria extracellularly during degranulation. Mast cells were stained with LysoTraker (left panels) and MitoTraker (middle panels). White rectangles in middle panels show mitochondria outside the cells. The merged panels show that the mitochondria had translocated to the cell surface where granules were undergoing exocytosis.

FIG. 7 illustrates mtDNA detection in the supernatant fluid from SP-stimulated LAD2 cells. LAD2 cells were treated with SP 2 μm for the indicated concentrations for 30 minutes. Supernatant fluids from both stimulated and control LAD2 cells were collected and assayed for (A) mtDNA, (B) Cyt C and (C) ATP. Cell viability (D) was measured by Tulubin-Blue staining. (n=3; *p<0.05, **p<0.01 compared to control). In FIG. 7A, Mt-7s and Mt-CytB gene expression in the supernatant fluid were quantitated by qPCR using the probe Hs 02596851_s1 (Applied Biosystems, Carlsbad, Calif.) and compared to control cells.

FIG. 8 illustrates that extracellular release of mtDNA is partially stored in exosomes. LAD2 cells were treated with SP (2 μm) for 30 min. Quantative PCR was performed to measure mtDNA level in with or without DNAase treated supernatants (FIG. 8A). Exosome-containing mtDNA level in LAD2 cells stimulated by SP is compared with mtDNA isolated from whole supernatant fluids (FIG. 8B) (n=3; *p<0.05, **p<0.01 compared to control). Exosomes were isolated from supernatants by Differential Ultracentrifugation. mtDNA was then isolated from exosomes.

FIG. 9 illustrates that sonicated mitochondria from cells and supernatant fluid from degranulated mast cells can trigger human mast cell degranulation, as well as IL-8, TNF and IL-1 beta release. LAD2 cells were incubated with mitochondria isolated from mast cells for either 30 min or 24 hr. Supernatant fluids from different conditions were collected. (A) β-Hex was detected at 30 min, while (B) IL-8, (C) TNF and (D) IL-1 beta were detected at 24 hr (n=3; *p<0.05, compared to control).

FIG. 10 illustrates that mt component activation of LAD2 cells is P2X7-receptor-dependent. LAD2 cells were pre-treated with P2X7 receptor inhibitor for 30 min and then stimulated with mt components. Different concentration of CpG-ODN [ISOLATED fomyl-peptides?] was used to stimulated LAD2 cells (FIG. 10A). LAD2 cells were treated with a P2X7 receptor inhibitor (FIG. 10B). Beta-hex release was measured 30 mins after stimulation. (n=3; *p<0.05, compared to control). FIG. 10C: live LAD2 cells were stained with stained with Fura-2to measure intracellular calcium changes in response to different concentrations of mt components.

FIG. 11 illustrates that sonicated mitochondria from cells and supernatant fluids from degranulated mast cells activate human keratinocytes to release IL-8 and VEGF, and human microvascular endothelial cells to release VEGF and TNF. HaCat and HMVEC cells were incubated with mitochondria isolated from mast cells for 24 hr. Supernatant fluids from different conditions were collected. Cytokines release from HaCat cells (A) IL-8, (B) VEGF and from HMVECs (C) VEGF and (D) TNF were detected at 24 hr (n=3; *p<0.05, compared to control).

FIG. 12 is a diagrammatic representation of proposed events following mast cell stimulation by SP leading to calcium influx, calcineurin activation, and Drp1 dephosphorylation. This process promotes mitochondrial fission, translocation to the cell surface close to granules undergoing exocytosis, and release of mtDNA.

FIG. 13 consists of scattergrams showing serum levels of (A) mt-Cytochrome B DNA and (B) mt-7S DNA in autistic patients (n=20; 16 males and 4 females; mean age 3.0±0.4 years) and controls (n=12; 11 males and 1 female; mean age 3±1.2 years). FIG. 13C is a scattergram showing serum levels of anti-mt antibodies type 2 (AMA-M2) in autistic patients (n=14; 11 males and 3 females; mean age 3.0±0.4 years) and controls (n=12; 11 males and 1 female; mean age 3±1.2 years). Genomic DNA for GAPDH was undetectable, excluding the possibility of cell contamination. The horizontal lines indicate the means.

FIG. 14 illustrates serum levels of (A) mtDNA Cytochrome B (CytB) and (B) mtDNA 7s in psoriatic patients (n=26) and controls (n=27). Genomic DNA GAPDH was undetectable, excluding the possibility of cell contamination. The horizontal lines indicate the means. (C) Linear regression analysis showing strong correlation between CytB and 7S.

### DETAILED DESCRIPTION OF THE INVENTION

Unless defined otherwise, all technical and scientific terms used herein have the same meaning as is commonly understood by one of skill in the art. The following definitions are provided to help interpret the disclosure and claims of this application. In the event a definition in this section is not consistent with definitions elsewhere, the definition set forth in this section controls.

### Definitions:

As used herein, the term “biomarker” refers to a molecule that allows for the detection of a biological state such as a diseased state.

Terms such as “treating” or “treatment” or “to treat” or “alleviating” or “to alleviate” as used herein refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. A subject is successfully “treated” according to the methods of the present invention if the patient shows one or more of the following: relief of one or more symptoms associated with the specific disease or disorder; reduced morbidity and mortality; and improvement in quality of life.

As used herein, the terms “inhibiting”, “to inhibit” and their grammatical equivalents, when used in the context of a bioactivity, refer to a down-regulation of the bioactivity, which may reduce or eliminate the targeted function, such as the extracellular secretion of mitochondrial DNA. In particular embodiments, inhibition may refer to a reduction of about 20%, 30%, 40%, 50%, 60%, 70%, 80%, 90% or 95% of the targeted activity. When used in the context of a disorder or disease, the terms refer to success at preventing or significantly delaying the onset of symptoms, alleviating symptoms, or eliminating the disease, condition or disorder.

As used herein, the term “substantially complementary” refers to complementarity in a base-paired, double-stranded region between two nucleic acids and not any single-stranded region such as a terminal overhang or a gap region between two double-stranded regions. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, substantially complementary sequences can refer to sequences with base-pair complementarity of 100, 95, 90, 80, 75, 70, 60, 50 percent or less, or any number in between, in a double-stranded region.

### Abbreviations:

Mt=Mitochondiral, TNF=tumor necrosis factor, DAMPs=damage associated molecular patterns, SP=Substance P, si=silencing or small interfering, CytB=cytochrome B

Mitochondria are bacteria that became symbiotic with eukaryotic cells and are responsible for cellular energy production. Increasing evidence indicates that mitochondria are not static, but undergo dynamic fission-fusion reactions that permit them to travel throughout the cell. This endows mitochondria with the ability to buffer intracellular calcium ions at surface where T cells are presented with specific antigen. It was also recently shown that injury releases endogenous mitochondrial damage-associated molecular pathogens (DAMPs), which include mtDNA that can stimulate Toll-like receptors (TLR)-9 on polymorphonuclear leukocytes to release IL-8 (Hyde, et al. 2010, Semin. Cell Dev. Biol).

Mitochondrial components are not normally found outside cells because they are extremely immunogenic. Mitochondrial health is kept in check by autophagy when damaged or “spent out” mitochondria are destroyed internally. Elsewhere, exosomes, which are nanovesicles that have recently been shown to transfer eukaryotic mRNA, microRNA as well as DNA, have been shown to contain mtDNA at least in the case of Glioblastoma cells and astrocytes. (Guescini, et al. 2010, J Neural Transm. 117:1-4).

Mast cells are implicated in inflammatory diseases. Take psoriasis as an example, which is characterized by keratinocyte proliferation and inflammation, as well as increased levels of mast cells, the peptide Substance P (SP), TNF and VEGF (Harvima, et al., 2008, Arch. Dermatol Res 300:461-76; Christy, et al., 2007, J Immunol. 179:2673-79). Psoriasis is also associated with comorbid diseases, especially psoriatic arthritis. Mast cells are found in large numbers in all tissues mostly around blood vessels in the skin where they participate in allergic and inflammatory reactions through release of multiple mediators with potent vasodilatory, inflammatory and pruritic properties. Mast cells exist at an increased level in lesional psoriatic skin. Histamine increases vascular permeability and stimulates cutaneous sensory nerves contributing to pruritus. Psoriasis is also triggered or exacerbated by acute stress. Skin mast cells may have important functions as “sensors” of environmental and emotional stress, possibly due to direct activation by corticotrophin-releasing hormone (CRH) and related peptides. Mast cells, and their circulating counterparts basophils, secrete numerous vasodilatory and pro-inflammatory mediators, such as histamine, kinins, proteases and TNF (preformed), as well as leukotrienes, prostaglandins, IL-1, IL-4, IL-5, IL-6, IL-8, IL-13, TNF and vascular endothelial growth factor (VEGF), which can also induce dilation of microvessels (Mousli, et al. 1994, Immunopharmacol 27:1-11).

Neuropeptides can trigger mast cell secretion. One of these neuropeptides is Substance P (SP), which was originally isolated and characterized from the brain, but later found to be widely distributed in many tissues, including epithelial and immune cells, where it is implicated in inflammatory processes (O'Connor, et al. 2004, J Cell Physiol 201:167-180). SP is released under stress. In particular, SP reactive fibers are localized close to mast cells from where SP could stimulate mast cell secretion in vivo (O'Connor et al., 2004). SP mediates inflammation, partially through mast cell activation (Remröd, et al. 2007, Arch Dermatol Res 299:85-91). Neuropeptides, especially SP, are involved in the pathogenesis of psoriasis (Jiang, et al. 1998, Int. J. Dermatol. 37:572-74).

In the present invention, we showed the role that mitochondria play in immune responses especially mast cell-effected inflammation. In particular, our data showed that mitochondrial fission, along with ensuing translocation and content release, in particular, mtDNA and ATP release, is required in the mast cell's degranulation process, which secretes pre-stored TNF. The discovery, with further supporting data, lead to the invention of methods, kits and compositions that are used to diagnose and/or treat various diseases, especially those implicated with abnormal immune activities such as autoimmune, inflammatory or neurodegenerative diseases.

Degranulation Induces Mitochondrial Fission in Human Mast Cells

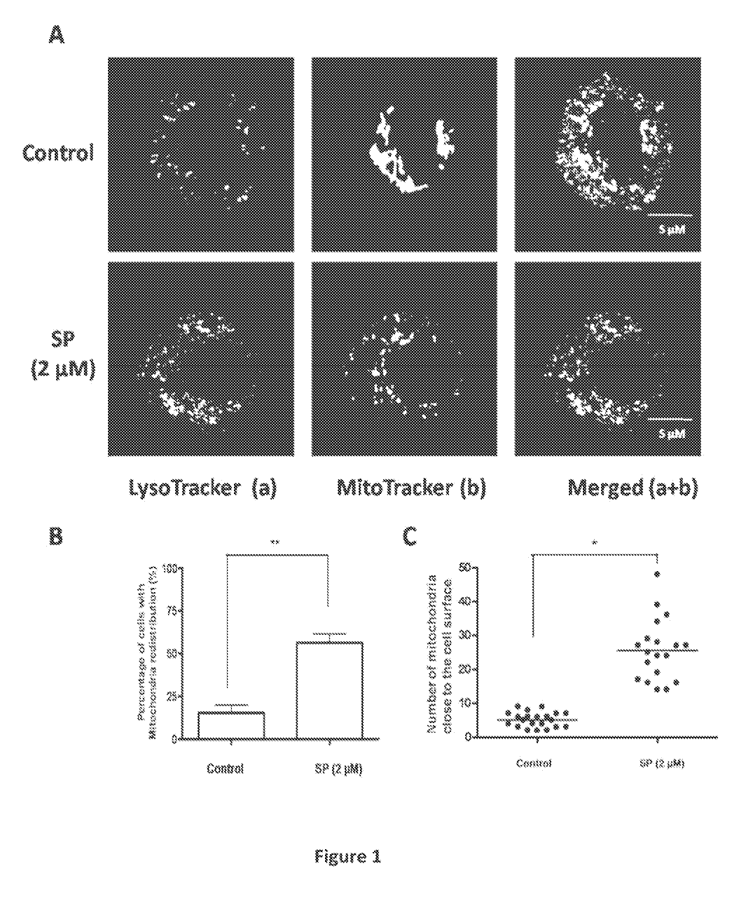

Examination of mitochondrial morphology of live LAD2 cells (human mast cells derived from a leukemia patient) and hCBMCs (human umbilical cord-derived cultured mast cells) by Confocal microscopy shows that in resting mast cells, mitochondria appear to have tubular-like or round shapes and are tightly gathered around the nucleus as a “mitochondria pool” (FIG. 1A). Very few mitochondria could be found in the cell surface region. It is almost impossible to distinguish individual mitochondria from this pool by confocal microscopy. Since the average pH of mast cell secretory granules is 5.5, LysoTracker (left panels) was used as a dye to stain the granules. After SP (2 μM) stimulation for 30 min at 37° C., mast cells underwent rapid degranulation, during which mitochondrial particles translocate throughout the cell (FIG. 1A). Quantification analysis of mitochondrial translocation shows that about 55% of SP-stimulated LAD2 cells contain mitochondria undergoing translocation as compared to 20% in control cells (FIG. 1B).

Further, the number of mitochondria close to the cell surface and outside the “mitochondria pool” was calculated using ImageJ software mitochondria counter plugin (Dagda, et al. 2009, J Biol Chem. 284:13843-55). Since cultured human mast cells do not attach to the culture flask, in order to minimize the effects that images are from different layers, all images were taken at the layer with the largest nucleus area. The number of mitochondria close to the cell surface showed significant increase after SP stimulation (FIG. 1C). Cell viability measured after 2 hours was more than 95% in LAD2 cells stimulated by SP, indicating that the mitochondrial fission is not due to apoptosis or necrosis. Cell viability was measured using trypan blue exclusion method where only dead cells would take up the trypan blue dye.

Mast Cell Degranulation and Pre-Stored TNF Secretion Require Drp1-Dependent Mitochondrial Translocation

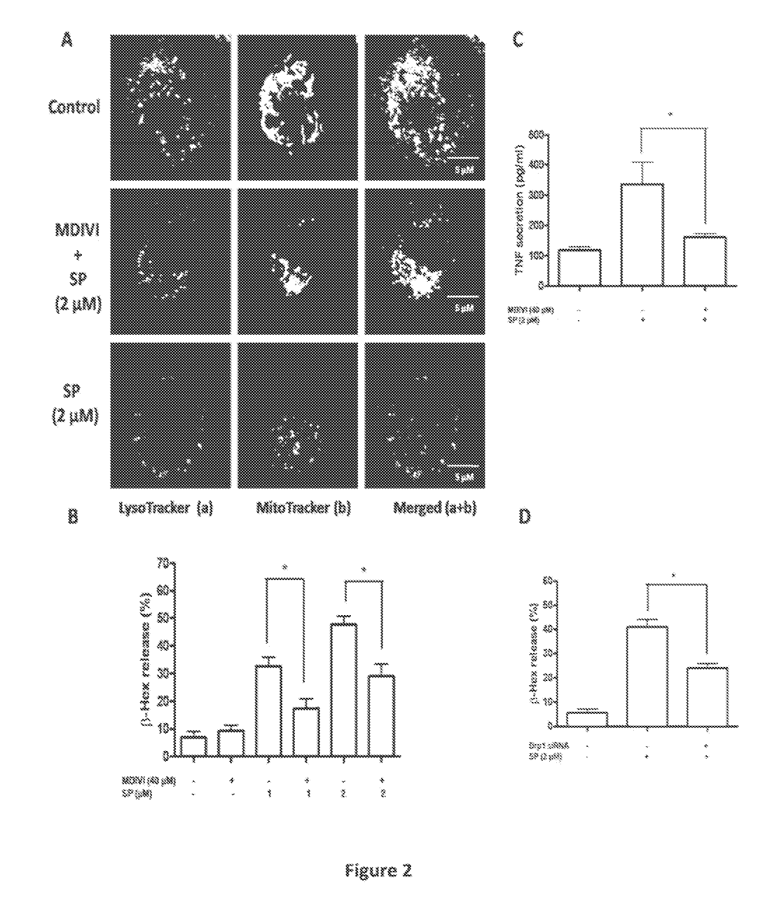

In order to rule out the possibility that mitochondrial translocation at the cell surface is a bystander effect secondary to cell shape changes during degranulation, the function of Drp1 in mast cell degranulation was investigated. Pretreatment of LAD2 mast cells with the mitochondria] Drp1 inhibitor MDIVI-1 at 40 μM for 15 min at 37° C. (Cassidy-Stone, et al. 2008, Dev. Cell 14:193-204) inhibited mitochondrial translocation (FIG. 2A) despite addition of SP (2 μM for 30 min). Moreover, MDIVI-1 pretreatment significantly inhibited SP-triggered β-hexosaminidase release and secretion of pre-stored TNF compared to cells stimulated by SP alone at both 1 and 2 μM (FIGS. 2B and 2C). Pretreatment of cells with another Drp1 inhibitor, Drp1 siRNA, also showed significant inhibition of SP-triggered β-hexosaminidase release (FIG. 2D).

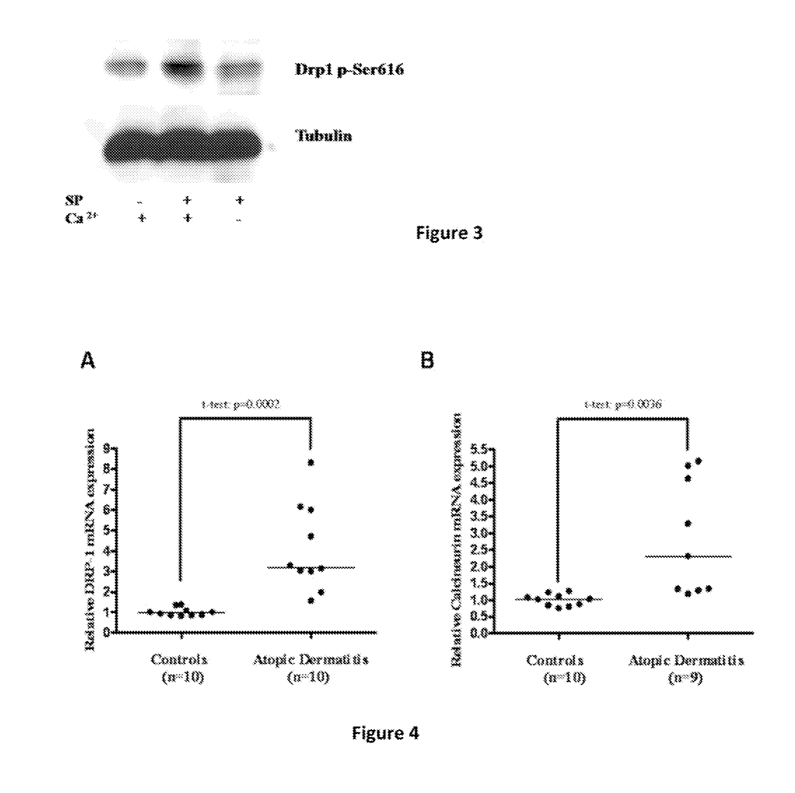

To confirm the involvement of Drp1, the State of phosphorylation of Drp1 at the amino acid serine-616 (Ser-616) was investigated through western blot analysis. FIG. 3 indicates that treatment of LAD2 mast cells with SP (2 μM for 30 min) lead to Drp1 phosphorylation, another piece of evidence that SP stimulation works through, at least partly, activation of Drp1.

Taken together, these data indiate that inhibitors of Drp1 function can be used as a means to treat diseases and conditions that implicate mast cell-effected immune responses.

Drp1 and Calcineurin Expression in Atopic Dermatitis (AD)

Analysis of skin biopsies from AD and normal skin controls reveals that gene expression of both Drp1 (FIG. 4A, controls n=10, patients n=10) and calcineurin (FIG. 4B, controls n=10, patients n=9) increased significantly in AD patients as compared to the control population.

SP-Stimulated TNF Secretion Involves Mitochondria Translocation

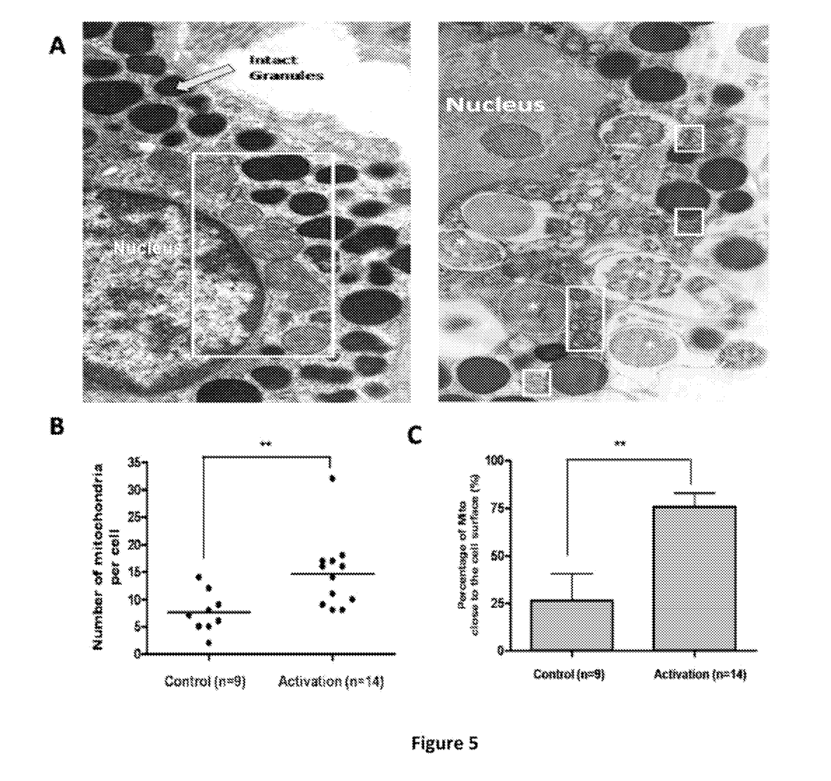

Observations using Transmission Electron Microscopy (TEM) (Theoharides, et al. 1998, Endocrinology 139:403-13) indicate translocation of mitochondria particles to sites of exocytosis. Mitochondria of unstimulated human skin mast cells are located around the cell nucleus and appear intact (FIG. 5A, left side). On the contrary, mitochondria of activated mast cells are much smaller than mitochondria from control cells and are located close to secretory granules undergoing exocytosis (FIG. 5A, right side). As shown in FIG. 5B, the number of mitochondria visible under the electron microscope increased significantly in SP-activated skin mast cells. And FIG. 5C shows a 200% increase from 25% to 75% in the percentage of mitochondria that have translocated close to cell surface. These results indicate that mitochondrial translocation to the cell surface is not a phenomenon of cultured mast cells, but characterizes activated human mast cells in diseases.

SP-Stimulated Mast Cell Secretion is Associated with mtDNA and ATP Release

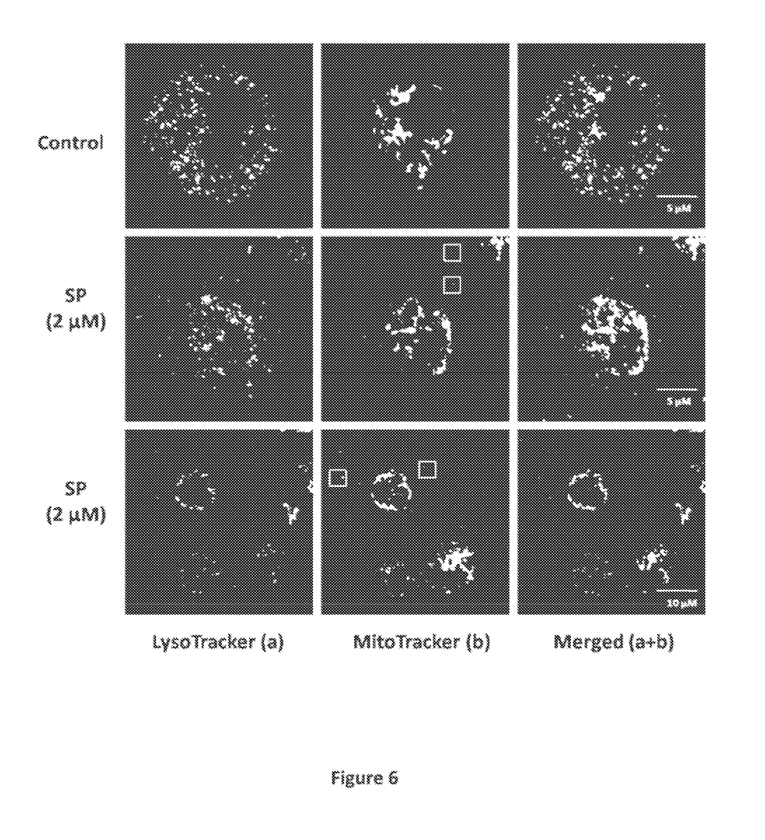

During LAD2 cells degranulation, mitochondria are detected extracellularly (FIG. 6). Surprisingly, many MitoTracker-stained particles are detected outside the cells (FIG. 6, middle panels, white rectangle), indicating the presence of functional mitochondrial particles.

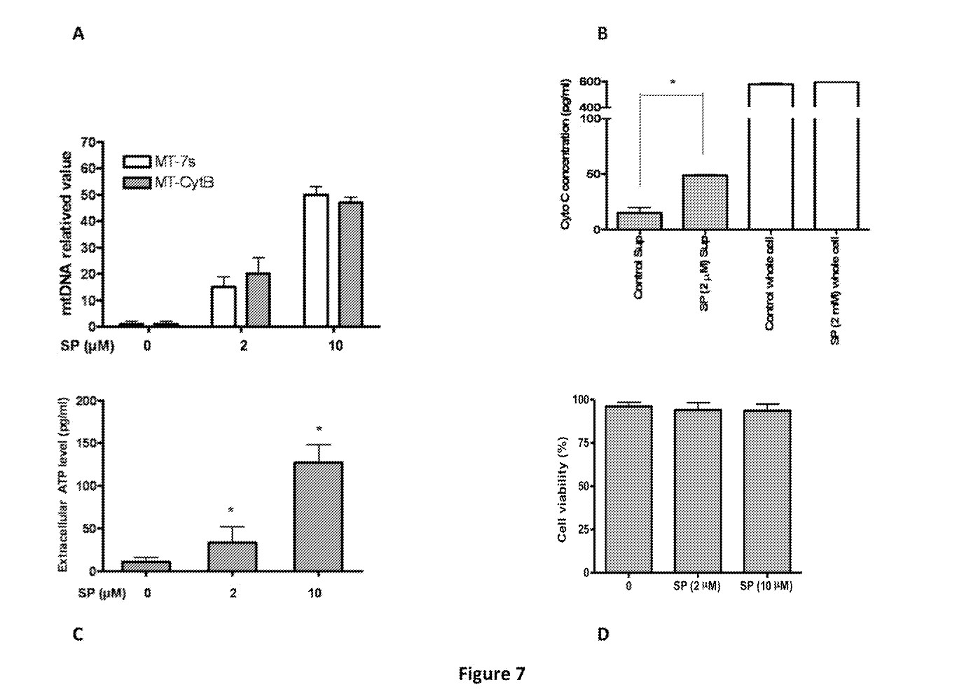

Supernatant fluid from LAD2 cells stimulated with SP (2 and 10 μM, respectively) for 60 minutes was found to contain at least two mtDNA species: 7s and Cytochrome B (CytB). The amount of these two genes, MT-7s DNA and MT-CytB, which are unique to mtDNA and not present in regular human genomic DNA, increased 200 times in SP-stimulated cells (10 μM SP) compared with that in the control as detected by real-time PCR (FIG. 7A). In contrast, genomic DNA for glyceraldehyde 3-phosphate dehydrogenase (GAPDH) is not detected (Hu, VIC TAMRA, data not shown). GAPDH is often stably and constitutively expressed at high levels in most tissues and cells, and therefore the absence of its DNA in the sample indicates that the extracellular presence of mtDNA is not due to cell death.

Further, cell viability found in supernatant fluids from stimulated LAD2 cells is about 99% (FIG. 7D). Also, both the ATP level (FIG. 7C) and Cytochrome C (Cyt C) (FIG. 7B) in the fluids showed marked increase, and in the case of ATP, also dosage-dependency on SP.

Extracellular Release of mtDNA from Mast Cells is Partially Stored in Exosomes

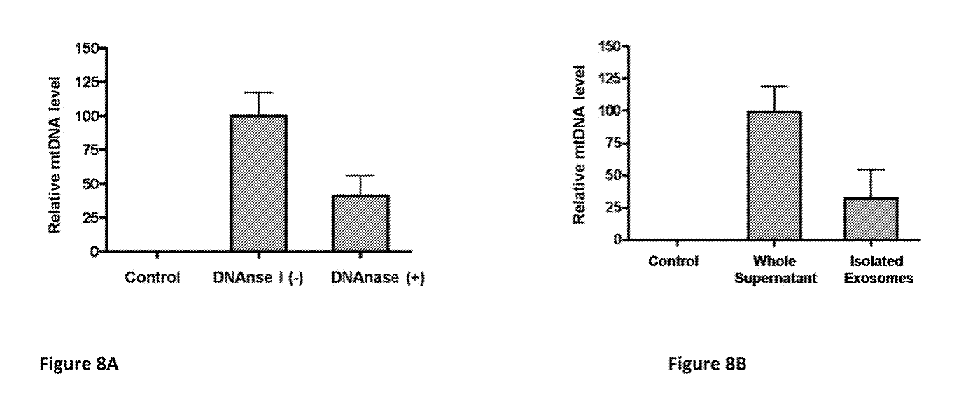

Exosomes from LAD2 cells treated with SP (2 μm) were collected by differential ultracentrifugation of the supernatants. These exosomes were further analyzed for the presence of mtDNA by isolating DNA and quantifying it through PCR. Results indicate that exosomes do contain mtDNA (data not shown), but not genomic DNA for GAPDH. To investigate if all of the extracellular mtDNA is confined inside the exosomes rather than absorbed on the pellet during ultracentrifugation, exosome samples were treated with DNaseI after resuspension in PBS. Such treatment lead to a significant decrease in mtDNA level as compared to control (FIG. 8A), indicating that a large portion of extracellular mtDNA is free.

When the total amount of extracellular mtDNA was compared with mtDNA contained in exosomes through the use of real-time PCR, the results indicate that only 25% of the mtDNA secreted by mast cells is contained in exosomes (FIG. 8B), and about 75% would be free. The possibility that mtDNA may be located in extracellular traps was also investigated. However, no such structure was detected in stimulated LAD2 cells (data not shown).

Mitochondrial Components Act as Autocrine Stimuli to Trigger Degranulation and Inflammasome Activation

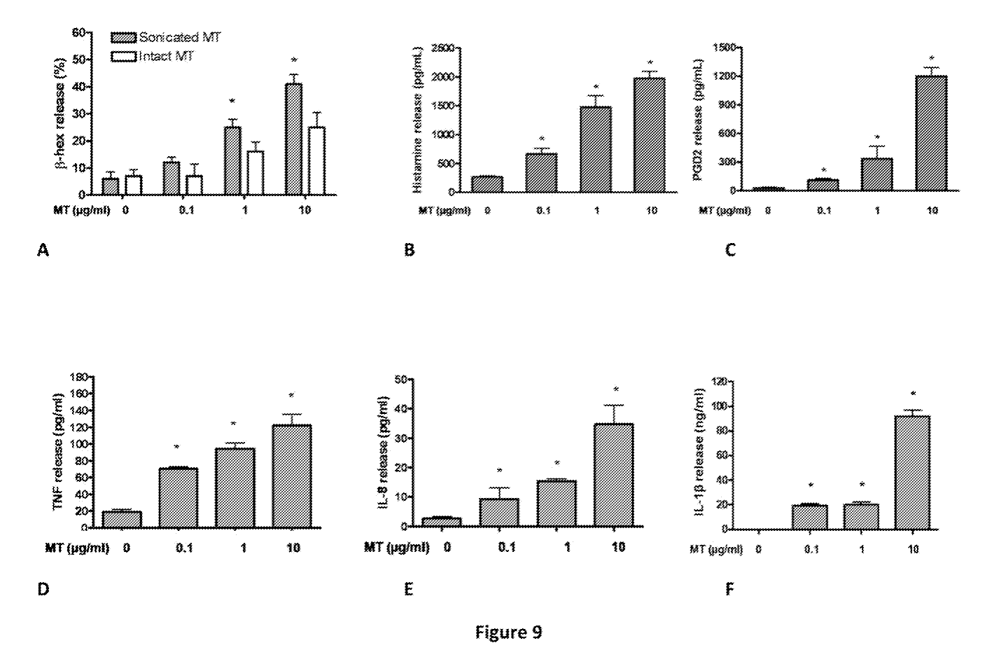

Mitochondria were isolated from either cultured human sarcoma cells or LAD2 mast cells, and then either used intact or after sonication to release all inner components. Mitochondrial concentrations larger than 0.1 μg/ml triggered mast cell degranulation resulting in cellular release of β-hexosaminidase (FIG. 9A), histamine (FIG. 9B) and PGD2 (FIG. 9C) within 30 minutes. Cells treated with sonicated mitochondria exhibited higher level of β-hexosaminidase secretion than those treated with intact mitochondria (FIG. 9A), suggesting that the release of some intra-mitochondrial components lead to higher potency in triggering degranulation. Mitochondria from both sarcoma and LAD2 cells showed similar effects (data not shown), indicating that the observed mitochondrial action likely did not depend on the cellular source of mitochondria.

The ability of mitochondrial components to stimulate de novo cytokine synthesis from LAD2 cells was also tested. LAD2 cells incubated with sonicated mitochondria for 24 hours produced significant amount of TNF and IL-8 (FIGS. 9D, E, respectivley). IL-lbeta secretion was also detected from LAD2 cells stimulated by mitochondrial components (FIG. 9F), indicating that mt components can activate inflammasomes.

ATP is a Key Component in Isolated Mitochondria Involved in Activation of Human Mast Cells

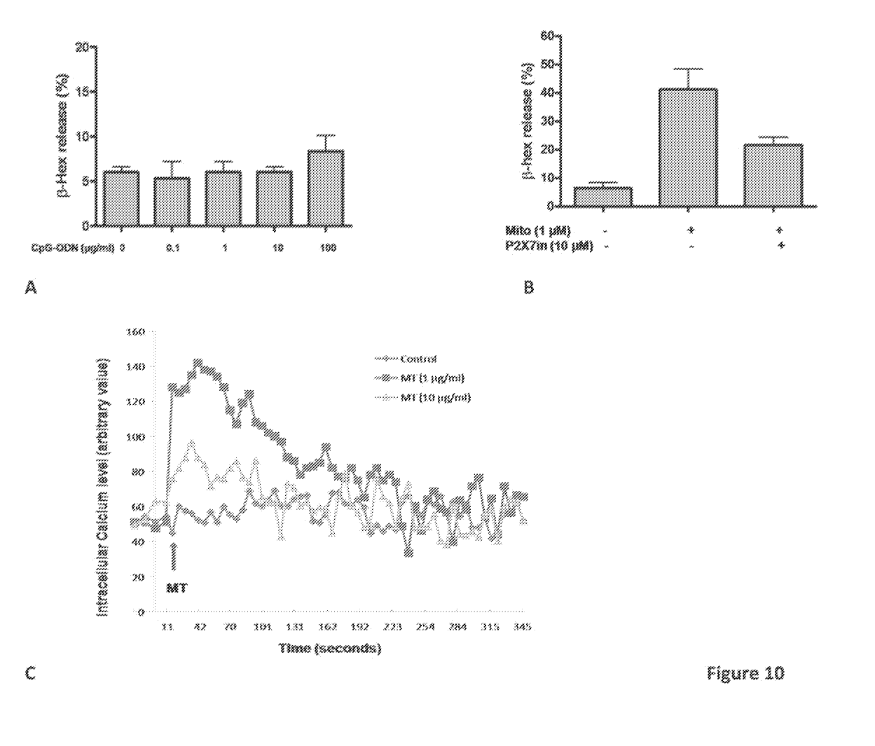

Several mitochondrial components were examined to see if they are responsible for triggering mast cell degranulation. Mitochondrial release would liberate fomyl-peptides, which are included in mitochondrial DAMPs. However, degranulation was not detected when isolated fomyl-peptides was used to stimulate both LAD2 (FIG. 10A) and hCBMCs cells (data not shown).

We then investigated which components from sonicated mitochondria may activate LAD2 cells. ATP has been reported to stimulated human mast cell degranulation (Cohen et al., 2001, Biochemistry 40:6589-97). Therefore, we tested if inhibition of P2X7 receptors, the primary receptor of ATP, can block the effects of mitochondrial components. As shown by data represented in FIG. 10B, pre-incubation of LAD2 cells with a P2X7 receptor inhibitor, A-438079 hydrochloride hydrate (commercially available from Sigma-Aldrich), partially blocked mitochondria-induced degranulation by 40%, indicating that ATP contained in mitochondria is at least partially responsible for mast cell degranulation.

We also investigated if mt components are able to increase intracellular calcium levels in mast cells. As FIG. 10C shows, isolated mt components were able to trigger immediate intracellular calcium increase in LAD2 cells.

Mitochondrial Components Act as Paracrine Stimuli and Trigger Cytokine Release

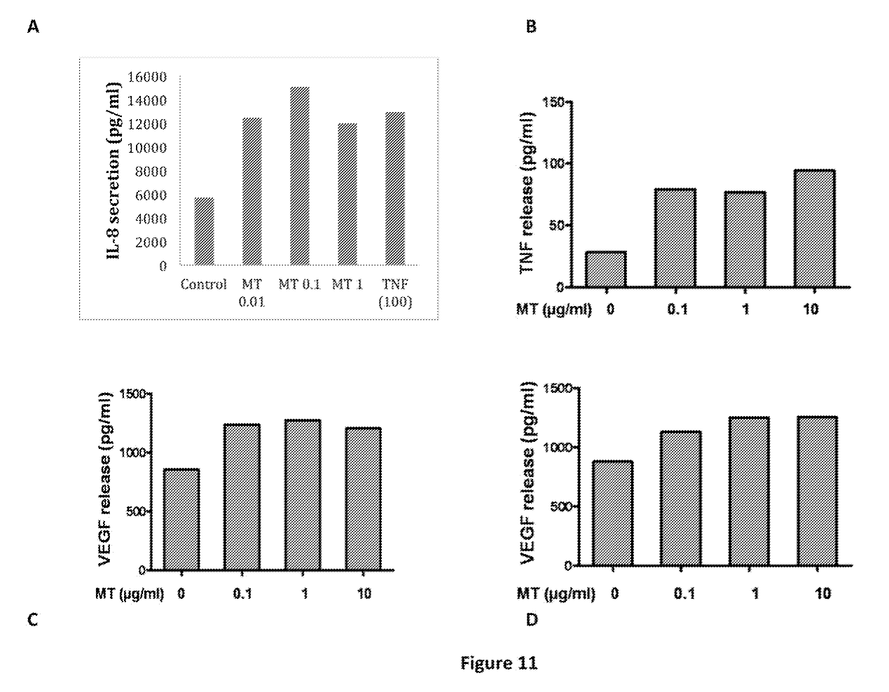

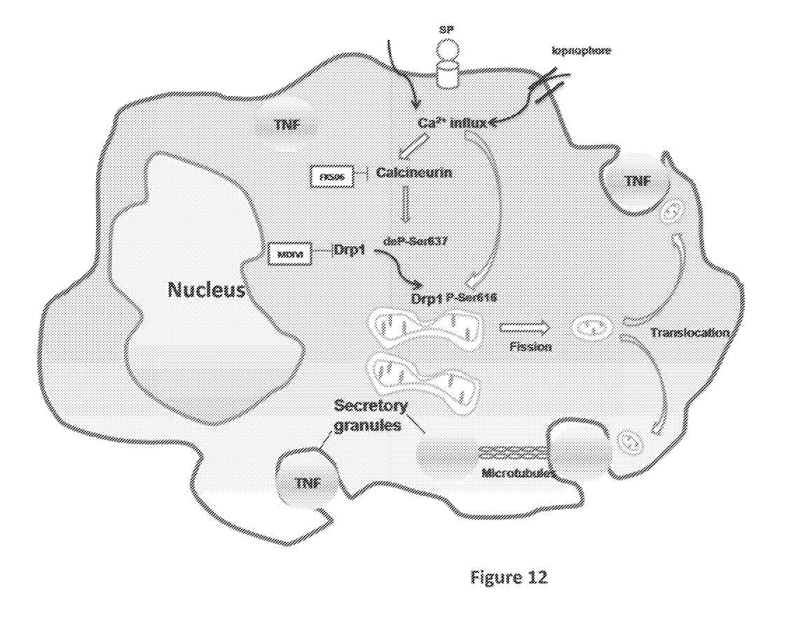

Mast cells in the skin are located close to keratinocytes and endothelial cells. IL-8 and VEGF, two of the most important cytokines of keratinocytes, are significantly elevated in the supernatant fluid after HaCat cells (human keratinocytes) were incubated with sonicated mitochondria isolated from LAD2 mast cells for 24 hours (FIGS. 11A, B).

We also investigated whether mitochondrial components could stimulate human microvascular endothelial cells (HDMEC). Primary HDMEC stimulated with sonicated mitochondrial components release significant amount of VEGF (FIG. 11C) and TNF (FIG. 11D).

The data above indicate that SP stimulates pre-stored TNF secretion from human mast cells through degranulation that requires mitochondrial fission as shown by significant inhibition of degranulation through the use of the mitochondrial fission inhibitor MDIVI-1. Confocal microscopy showed mitochondrial translocation close to sites at the cell surface near where secretory granules are undergoing exocytosis. It is hypothesized that mitochondrial fission and translocation may be necessary for providing energy locally for the secretory granules to fuse with the cell membrane required for degranulation. In activated β-pancreatic cells, mitochondria undergo fission during insulin secretion from storage vesicles. Alternatively, mitochondria may translocate to the cell surface to buffer local increases in calcium ions required for exocytosis. Mitochondrial shape changes and translocation to the cell surface were also shown to be involved in T cell activation and chemotaxis.

Stimulation of LAD2 (Kirshenbaum, et al. 2003, Leuk Res 27:677-682) mast cells by SP (1 or 2 μM for 30 min) activates degranulation of pre-stored TNF (Kulka, et al. 2007, Immunology 123:398-10). At concentrations similar to the ones used here, SP concentrations higher than 1 μM were reported to be able to enhance the rate of oxygen consumption of isolated cardiac cell mitochondria. SP (0.01 to 1 μM) was reported to induce selective gene expression and release of TNF, but not of IL-1, IL-3, IL-4, IL-6 or GM-CSF from a murine mast cell line and isolated murine peritoneal mast cells. With respect to human skin mast cells, SP (0.8-100 ηM) induced histamine and de novo synthesized TNF release. Moreover, SP (0.3-30 μM) selectively induced only de novo TNF gene expression and release, unlike FcεRI crosslinking that induced gene expression of IL-4, 1L-5 and TNF (Okayama, et al. 1998, Int. Arch. Allergy Immunol. 117 Suppl 1:48-51). Nevertheless, none of these papers investigated or reported mitochondrial translocation to the cell surface.

Our data also showed that Drp1, which is necessary for mitochondrial fission, is expressed at high levels in LAD2 cells activated by SP, as well as in skin biopsies from AD patients. Additionally we showed that gene expression of calcineurin is increased in the skin of AD patients. Dephosphorylation of Drp1 by calcineurin is required for recruitment of Drp1 to the mitochondria leading to increased mitochondrial fission. The chronic inflammation of the skin in AD is often the first sign of atopic disease (Simpson. E. L. 2010. Atopic dermatitis: a review of topical treatment options. Curr. Med. Res. Opin. 26:633-640). AD involves mast cells (Kawakami, et al. 2009. Curr. Opin. Immunol. 21:666-78) and is often not helped by antihistamines, implying release of other pruritic molecules, such as TNF. It is, therefore, of interest that one of the most effective treatments of AD is the calcineurin inhibitor, tacrolimus (FK506) (Simpson, 2010). FK506 had been reported to inhibit secretion of IgE-induced histamine from the same granules containing preformed TNF in rat mast cells (Sengoku, et al, 2000. Int J Immunopharmacol 22:189-201). FK506 was also reported to deplete SP from peripheral sensory neurons (Inagaki et al. 2010, Eur. J Pharmacol. 626:283-289) and prevent pruritus in a mouse model of AD (Inagaki et al., 2010). Our findings with SP stimulation of pre-stored TNF secretion also provide a possible explanation for increasing evidence that anti-TNF therapy is useful in severe cases of AD (Rigopoulos, et al. 2008. Expert. Opin. Biol. Ther. 8:123-33; Rullan, et al. 2009, J Drugs Dermatol. 8:873-76; Sockolov, et al. 2009, J Dermatolog. Treat. 20:319-327).

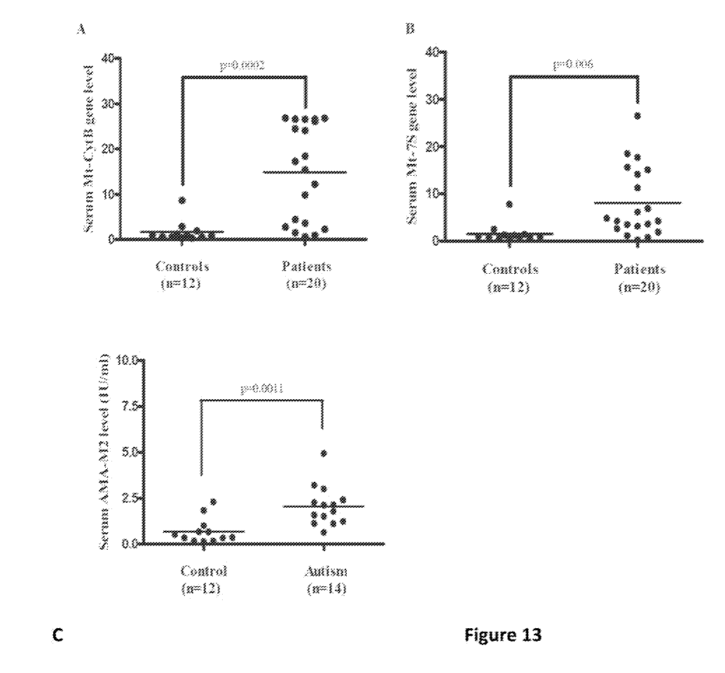

An important new finding through the present experiments is that the secretion into extracellular space of mt components including mtDNA and ATP after a mast cell is stimulated with SP (FIG. 13). These mt components then stimulate mast cells, keratinocytes and endothelial cells to produce proinflammtory cytokines. Extracellular mtDNA release was reported following TNF-induced murine embryonic fibroblasts (Nakajima, et al, 2008, J Biol. Chem. 283:24128-35) but this was accompanied by caspase-dependent cell death. Moreover, Damaged Associated Mitochondrial Patterns (DAMPs) were purified from blood from patients with trauma-induced sepsis in the absence of any apparent infection (Zhang et al. 2010, Nature 464:104-07), but these are also derived from dead cells due to trauma. One paper reported the release of mtDNA extracellular traps in response to infection triggers (Yousefi et al., 2009). However, in that case, the release was limited to neutropils and was also only associated with a trigger of infectious origins. We did not detect any extracellular traps in our experiments, indicating our mitochondrial release is different from extracellular trap formation.

More recently, it was reported that astrocytes and glioblastoma cells could release mtDNA inside exosomes (Guescini et al., 2010). Exosomes can transport RNA from human mast cells as well as DNA, mRNA and microRNA from Jurkat T cells and promyelocytic cell lines. However, these nucleic acids are of eukaryotic origin and are shielded inside membrane-bound nanovesicles. We have now confirmed that some released mtDNA indeed are stored in exosomes, but most of the mtDNA released by mast cells appear to exist and function outside exosomes. Some of the free mtDNA will enter into vascular circulation and most likely the majority of released mtDNA will be delivered to the lymph nodes where it activates other immune cells such as dendritic cells (Willart et al., 2008, Clin Exp. Allergy, 39: 12-19), as recently shown for mast-cell-derived-TNF-containing “particles” (Kunder, et al., 2009, J Exp Med. 206:2455-67).

We also disclose that mast cell degranulation results in ATP release and a significant part of the stimulant effect of extracellular mt components is blocked by an ATP receptor P2X7r antagonist, suggesting that nit ATP plays a critical role in generating immune responses. Extracellular ATP was shown to trigger and maintain inflammation in asthmatic airway (Bromm, et al. 1991, Rev. Neural (Paris) 147:625-43). Delivery of ATP in mt particles will certainly permit it to survive longer and affect cells at some distance for its original source.

The ability of mast cells to be activated by mt components to release 1L-8, VEGF and IL-1beta indicates their ability to activate the inflammasomes (Feldmeyer, et al. 2010, Eur. J Cell Biol. 89:638-644) and TLRs on mast cells. Mast cells express TLRs, including TLR9 that can be activated by bacterial DNA sequences, activation of which leads to release of different cytokines (Bischoff, et al. 2007. Immunol Rev. 217:329-37). However, we disclose for the first time that nit components can activate mast cells and show that mitochondrial fission and translocation is necessary for mast cell degranulation while stimulated by both allergic and non-allergic triggers. The fact that mitochondrial components can also stimulate keratinocytes and microvascular endothelial cells implies that they may be involved in the pathogenesis of skin inflammation which is characterized by neovasculization and keratinocyte hyperproliferation (Schon, et al. (2005) N. Engl. J Med 352:1899-12). Mast cells have been implicated in psoriasis and we recently showed that SP and IL-33 are important in psoriasis (Theoharides, et al. (2010) Proc Natl Acad Sci USA 107:4448-53).

Mast cells are involved in virtually all inflammatory diseases including asthma. rheumatoid arthritis, and inflammatory bowel diseases. Mast cells are also one of the most important effector cells in the skin responding to environmental and infectious insults. All of these diseases are strongly modulated by stress and a role of neuropeptides including SP appears likely (Theoharides, et al. 2004. J Neuroimmunol 146:1-12). Therefore, the mechanisms described here appear to be most relevant to a number of important diseases. Based on the unique, and, heretofore, unrecognized function of mitochondria in mast cell activation, the present invention provides new diagnostic and treatment options for inflammatory, autoimmune diseases, and neurodegenerative diseases.

The present invention also includes data (in Example section below) specifically showing detection of mtDNA in the serum of patients with ASD and psoriasis. This is the first time that mitochondrial components are shown to be released from activated normal cells, as well as in patients with an inflammatory skin disease or ASD. It is important to note that mothers with psoriasis during pregnancy had a higher chance of delivering children who developed autism later (Croen, et al. (2005) Arch. Pediatr Adolesc. Med 159:151-157).

Release of mitochondrial components opens the door to a number of diagnostic and therapeutic approaches. Serum mitochondrial components, mtDNA, ATP, inhibitors of various mt components and anti-mitochondrial antibodies can he utilized in these approaches.

As described above, a first aspect of the present invention relates to methods, reagents and kits for diagnosing various autoimmune, inflammatory or neurodegenerative diseases through the detection of at least one extracellular mitochondrial component or its antibody as a biomarker for the disease in a patient's biological sample. In order to screen out false positives where extracellular mtDNA is released due to cell death, the diagnostic method of the invention optionally includes ways to confirm cell viability in the sample, e.g., by including in the kit of the invention, reagents capable of detecting any DAMPs released after apoptosis or necrosis as an indication of cell death or trauma. In another embodiment, an embodiment of the kit of the invention includes trypan blue dye and other means known in the art for verifying cell viability in a biological sample.

In various embodiments, a probe or detection means for a (first) mitochondria-specific marker is used according to the present invention for the diagnosis of the suspected disease(s). The marker can be one or more of the following: mitochondrial peptidoglycan, mtDNA, formyl-peptides, cytochrome c, ATP and/or an antibody against any mitochondrial component. In a particular embodiment, the probe for the marker includes a region of an mtDNA for hybridizing with a complementary region of extracellular mtDNA in the sample. In one example, the probe is constructed for detecting CpG-rich regions of mtDNA, a characteristic of the mitochondrial DNA. The probe may emit a detectable signal itself, e.g., florescence, radioactivity, chemoluminescence/chemiluminescence, and so on. Alternatively, the probe may require a second reagent for signal detection, as one in the art readily understands. These variations are contemplated by the present invention and not enumerated herein.

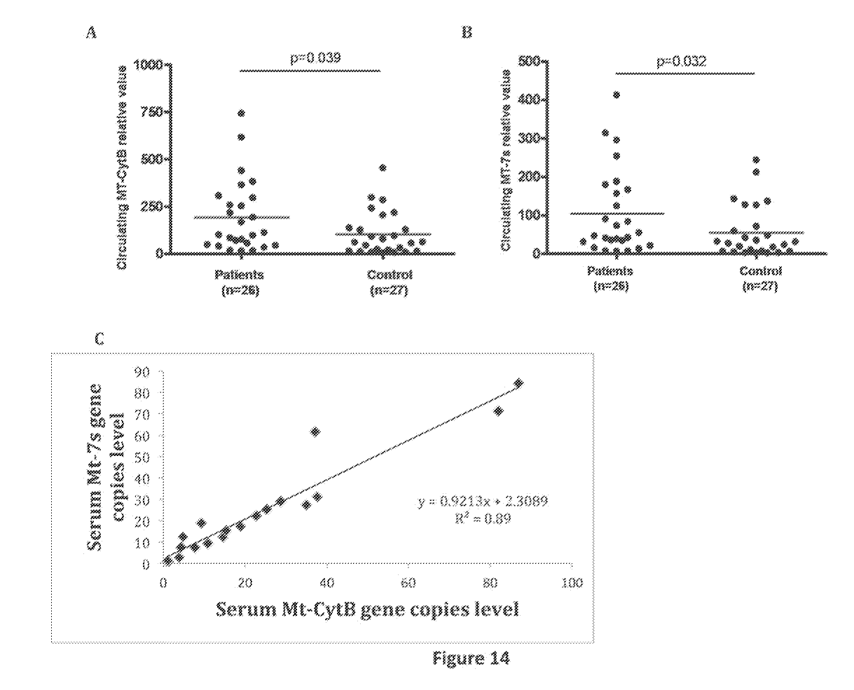

In one embodiment, detection of a serum level of mtDNA significantly, e.g., about 30%, 50%, 100%, 200% or even more, above those levels detected in a healthy population would lead to a diagnosis of having the suspected disease or condition. According to one study, a healthy level of extracellular mtDNA is quite low, at between about 1.0 to about 2.0 ng/mL (Zhang et al. 2010, Nature 464:104-07). According to our data, serum mtDNA level in autistic patients can be about 8-10 times of that of a healthy control group of similar ages (FIG. 13). Therefore, according to one embodiment, detection of about at least 5, 6, 7, 8, 9, or 10 times the amount of serum mtDNA compared to a healthy control group is used as a diagnostic threshold for autism and other neurodegenerative diseases. Our data also indicate that psoriatic patients can have about twice the amount of serum mtDNA compared to a healthy control group (FIG. 14), and therefore, in a further embodiment of the invention, detection of about at least 1.5 or 2 times the amount of serum mtDNA compared to a healthy control group is used as diagnostic threshold for psoriasis and similar inflammatory skin diseases.

In order to utilize such a probe, however, it is often necessary to first have enough genetic materials from the sample if the first marker is selected to be extracellular DNA or RNA from the mitochondria. Therefore, the kit of the present invention may also include reagents commonly used for isolating, exacting, purifying, reverse-transcribing, amplifying and/or quantitating genetic materials (DNA and/or RNA). In an exemplary embodiment, isolation reagents are first used on human serum samples to isolate (extract and purify) total DNA or RNA. There are many commercially available kits for this purpose, e.g., Qiagen DNA Micro extraction kit (Qiagen, CA) which includes DNA isolation reagents and DNA isolation columns. Next, quantitative PCR reagents including primers with sequences designed for amplifying a region of a mitochondrial DNA are used to measure copy numbers of mitochondrial-DNA-specific sequences, e.g., mt-7S and mt-Cytb, in the sample. There are also commercially available assays and kits for such purpose, e.g., Applied Biosystems' Taqman® qPCR kit which contains DNA amplification reagents and probes against ms-7S and mt-Cytb sequences.

In another embodiment, the probe for the mitochondria-specific marker comprises a probe for an anti-mitochondrial antibody (against at least one nit component), some of which are commercially available, e.g., DRG International Product No. EIA-3604 currently used for diagnosis of biliary cirrhosis associated with damaged liver cells. Elevated level of circulating mitochondrial proteins will trigger human immune response and produce antibodies against those proteins. Therefore, an elevated level of the resulting antibody is also a good surrogate indication that abnormal amounts of mitochondrial parts are circulating freely. In an embodiment, a protein detect kit (ELISA) commercially available from Cayman Chemical is used to detect the level of Anti-Mitochondrial Antibody 2 (AMA-M2). According to our data, serum AMA-M2 level in autistic patients can be about 5 times of that of a healthy control group of similar ages (FIG. 13C). Therefore, according to one embodiment, detection of about at least 3, 4, or 5 times the amount of serum AMA-M2 compared to a healthy control group is used as a diagnostic threshold for autism and other neurodegenerative diseases.

In addition to looking for the presence of a mitochondria-specific marker, the kit of the present invention may further include reagents commonly used for detecting the presence of a second marker in the sample. Inclusion of a second probe or detection means for the second marker enhances specificity of the diagnosis and reduces the chance of false positives. Such a secondary marker may be a known indicator for the suspected disease such as neuropeptides that are known to be associated with an autoimmune, inflammatory or neurodegenerative disease, e.g., neurotensin for autism and an anti-nuclear antibody (ANA) for autoimmune disease.

In a second aspect of the present invention, inhibition of release of mitochondrial components and/or inhibition of released mitochondrial components is used for prophylactic or symptomatic treatment of patients with autoimmune, inflammatory diseases and neurodegenerative diseases. In various embodiments according to this aspect, a therapeutic agent is devised based on its ability to inhibit the activation of at least one extracellular mitochondrial component activity that contributes to the mast cell secretion. For example, the agent can be an inhibitor of one or more of the following: extracellular mitochondrial DNA, extracellular mitochondrial peptidoglycan, ATP, a cellular receptor activated by mitochondrial DNA. In a particular embodiment, the agent comprises a DNA sequence substantially complementary to and capable of neutralizing extracellular mitochondrial DNA. Other agents capable of “neutralizing” the mtDNA are also contemplated as part of this inventive aspect. For instance, a viral DNA vaccine was produced with lasting immunity against the corresponding viral DNA (Hassett D E et al. J Virol, 74:2620-2627, 2000). A vaccine produced in a similar fashion against the mtDNA is contemplated by the present invention.

### EXAMPLE 1

### mtDNA Found in Serum of ASD Patients

One of the diseases that can be diagnosed and treated using the present invention is Autism. Autism Spectrum Disorders (ASD) are neurodevelopmental disorders characterized by varying degrees of dysfunctional communication and social abilities, repetitive and stereotypic behaviors, as well as attention, cognitive, learning and sensory deficits. There remain few if any clues regarding the underlying pathophysiology, early detection biomarkers or effective treatment for ASD.

A number of studies have suggested that ASD may be associated with some immune dysfunction in the patient or the mother during gestation. Additional evidence suggests that ASD may have a neuroimmune component (Theoharides, T. C., et al. 2009. Exp Opinion on Pharmacotherapy 10:2127-43), and it was recently shown that the peptide neurotensin (NT) is significantly increased in young children with autistic disorder (Angelidou A et al., 2010). A number of studies reporting mitochondrial (nit) dysfunction in autism have focused on altered energy metabolism (Palmieri, L. and A. M. Persico. 2010. Biochim. Biophys. Acta 1797:1130-1137), and concluded that it may involve a subset of children with autism (Weissman, et al. 2008. PloS One 3:e3815). Mitochondria are the primary energy-generating organelles in eukaryotic cells, and they participate in multiple intracellular processes, including calcium buffering. However, mitochondria are bacteria that became symbiotic with eukaryotic cells and are typically prevented from being released extracellularly by autophagy. In the case of ASD patients, however, mitochondrial components may have been released extracellularly early in life and induce an “autoimmune” response.

Investigation was carried out among a homogeneous group of young Caucasian children with the same endophenotype. Subjects were diagnosed with autistic disorder using the ADI-R and ADOS-G scales, which have been validated in the Greek population (Papanikolaou, et al. 2008. J Autism Dev Disord. 39:414-420) There were no apparent clinical differences, such as gastrointestinal problems or apparent mitochondrial dysfunction that may have allowed separation of the autistic patients in subgroups.

Blood was obtained in the morning at least 2 hours after breakfast to minimize any diurnal or postprandial effects. Serum samples from patients and controls were aliquoted and frozen at −80° C. until assayed. All children met 1CD-10 criteria for autistic disorder. The exclusion criteria included: (1) Any medical condition likely to be etiological for ASD (e.g. Rett syndrome, focal epilepsy, Fragile X syndrome or Tuberous Sclerosis); (2) Any neurological disorder involving pathology above the brain stem, other than uncomplicated non-focal epilepsy; (3) Contemporaneous evidence, or unequivocal retrospective evidence, of probable neonatal brain damage; (4) Any genetic syndrome involving the CNS, even if the link with autism is uncertain; (5) Clinically significant visual or auditory impairment, even after correction; (6) Any circumstances that might possibly account for the picture of autism (e.g. severe nutritional or psychological deprivation); (7) Active treatment with pharmacological or other agents; (8) Mastocytosis (including urticaria pigmentosa); (9) History of upper airway diseases; (10) History of inflammatory diseases; (11) History of allergies. The controls were normally developing, healthy children, unrelated to the autistic subjects, and were seen for routine health checkups in a location in Greece.

Total DNA was extracted from collected serum samples using Qiagen DNA Micro extraction kit (Qiagen, CA). Mitochondria-specific DNA for Cytochrome B (Mt-CytB) and 7S (Mt-7S) was detected and quantified by Real time PCR using Taqman assay (Mt-7S: Hs02596861_s1; Mt-CYB: Hs02596867_s1; GAPDH: Hu, VIC, TAMRA, Applied Biosystems, CA). GAPDH DNA detection was used to exclude genomic “contamination.” In addition to DNA detection, anti-mt antibody Type 2 (AMA-M2) from a commercial EIA Kit (DRG International, Germany) was also used to detect the presence of mt proteins in the samples.

Data show that serum samples collected from young autistic patients contained detectable or elevated levels of mtDNA for cytochrome B (p=0.0002) and for 7S (p=0.006) (FIGS. 13A and 13B). While it took, on average, about 26 PCR cycles to generate detectable amount of mtDNA for the control group, it only took about 22-23 cycles for most of the patient group, indicating a much greater amount in the starting copies for the patient samples. Accordingly, it might be a useful and practical threshold for a pathological diagnosis of a disease implicating extracellular mtDNA if it takes at least 2 fewer PCR cycles to generate detectable level of serum mtDNA in comparison to a healthy control group.

Serum samples of autistic patients also contained detectable or elevated levels of AMA-M2 antibodies against a Type 2 protein in mitochondria (p=0.001) (FIG. 13C). Normally, healthy children have AMA level lower than 0.5 IU per ml. The average AMA antibody level in autistic patients tested was about 2.5 IU per ml, about 5 times, on average, compared to unrelated, normal controls. Accordingly, at least about three times the normal amount of AMA-M2 antibodies or more than 1.5 or 2.0 IU per ml can be used as a threshold for a pathological diagnosis. This can be used alone or in combination with any other test proposed herein for diagnostic purpose. There was no presence of GAPDH DNA, indicating that there was no genomic DNA release.

Serum mtDNA has never been associated with any neuropsychiatric and neurodegenerative disease before. Moreover, anti-mt antibodies have been clinically detected only in primary biliary cirrhosis (Van de, et al. 1989, N. Engl. J Med. 320:1377-80). Consequently, our study compared the level of detection to a control group and found that serum mtDNA level in young autistic patients can be about 8-10 times of that of a healthy control group of similar ages (FIG. 13).

There is no reason to suspect that these extracellular mitochondrial components derived from apoptotic or necrotic cells because no GAPDH DNA was detected. Moreover, there was no apparent cell damage, at least outside the brain, in autistic patients. The mitochondrial components observed in the data might have been secreted by immune cells, as was recently reported for neutrophils activated by infectious triggers (Yousefi, et al. 2009, Cell Death. Differ. 16:1438-44). However, as mentioned earlier, this mitochondrial release was contained in “traps” close to the cell and was limited to the action of a trigger derived from pathogenic bacteria, not auto-immunity or inflammation or neuropeptide triggers involved in the present invention.

### EXAMPLE 2

### Psoriatic Patient Serum Contains Elevated mtDNA Compared with Healthy Controls

Psoriasis is a chronic inflammatory skin disease that involves keratinocyte, VEGF and mast cells. The presence of free mtDNA in the blood of psoriatic patients was investigated. Serum from 26 psoriatic patients contained significantly more mtDNA for CytB (p=0.0002) and 7S (p=0.006) compared to those of controls (n=27) (FIGS. 14A and 14B). Linear regression analysis shows an excellent correlation (R̂2=0.89) between CytB and 7S (FIG. 14C). No DNA for GAPDH was detected indicating there was no genomic DNA release due to cell death. Our data indicate that psoriatic patients can have about twice the amount of serum mtDNA compared to a healthy control group.

All publications described herein are incorporated by reference in their entirety to the full extent allowed by pertinent patent laws.

What is claimed is:

1. A method for diagnosing a disease, said method comprising the steps of:
detecting the presence of at least one extracellular mitochondrial component in a biological sample obtained from a patient as a mitochondria-specific marker indicative of said disease; and
confirming the absence of an indication of cell apoptosis or necrosis in the same biological sample.

2. The method of claim 1 wherein said disease is selected from the group consisting of an autoimmune disease, an inflammatory disease and a neurodegenerative disease.

3. The method of claim 2 wherein said autoimmune disease is selected from the group consisting of Churg-Strauss Syndrome, Coeliac disease, Hashimoto's thyroiditis, Goodpasture Syndrome, Graves’ disease, inflammatory bowel disease, psoriasis, psoriatic arthritis, rheumatoid arthritis (RA), Sjögren's syndrome and systemic lupus erythematosus (SLE); wherein said inflammatory disease is selected from the group consisting of allergy, amyotrophic lateral sclerosis (ALS), asthma, chronic inflammatory disorder, atopic dermatitis, coronary atherosclerosis, interstitial cystitis, diabetes mellitus type 1 (IDDM), idiopathic thrombocytopenic purpura, multiple sclerosis and chronic pancreatitis; and wherein said neurodegenerative disease is selected from the group consisting of autism spectrum disorders (ASD), chronic fatigue syndrome, chronic prostatitis, fibromyalgia, vitiligo and Parkinson's Disease.

4. The method of claim 1 further comprising using a second marker for said disease, wherein said second marker is selected from the group consisting of an antinuclear antibody (ANA) and neurotensin.

5. The method of claim 1 wherein said disease is selected from the group consisting of rheumatoid arthritis, psoriasis, autism spectrum disorders (ASD), SLE and mastocytosis.

6. The method of claim 1 wherein said mitochondrial component is selected from the group consisting of mitochondrial peptidoglycan, mitochondrial DNA, formyl-peptides, cytochrome c, and ATP.

7. The method of claim 1, wherein the confirmation step comprises setting out to detect any damage-associated molecular patterns (DAMPs) in the sample or to detect any cellular intake of trypan blue dye as indication of cell apoptosis or necrosis.

8. A diagnostic kit for diagnosing a disease, said kit comprising a reagent for detecting, in a biological sample obtained from a patient, the presence of at least one extracellular mitochondrial component as a mitochondria-specific marker for said disease.

9. The kit of claim 8 wherein said mitochondrial component is selected from the group consisting of mitochondrial peptidoglycan, mitochondrial DNA, formyl-peptides, cytochrome c, and ATP.

10. The kit of claim 8 further comprising an extraction reagent for isolating genetic materials from said biological sample from the patient, wherein said biological sample is selected from the group consisting of plasma, serum, urine, lymph, cerebrospinal fluid, colonic fluid, nasal fluid, vaginal secretion, saliva, sweat, skin biopsy and other tissue biopsy.

11. The kit of claim 8, further comprising reagents for conducting PCR, real time PCR or qPCR.

12. The kit of claim 8, further comprising a pair of primers comprising sequences configured for amplifying at least a region of a mitochondrial DNA, preferably comprising one or more CpG dinucleotides.

13. The kit of claim 8, further comprising a cell-viability-testing reagent for confirming the absence of an indication of cell apoptosis or necrosis in the same biological sample.

14. The kit of claim 8, further comprising a probe for detecting an antibody against at least one mitochondrial component.

15. The kit of claim 8, further comprising a probe for detecting neurotensin or antinuclear antibody (ANA).

16. The kit of claim 8, wherein said disease is selected from the group consisting of an autoimmune disease, an inflammatory disease and a neurodegenerative disease.

17. The kit of claim 16, wherein said autoimmune disease is selected from the group consisting of Churg-Strauss Syndrome, Coeliac disease, Hashimoto's thyroiditis, Goodpasture Syndrome, Graves' disease, inflammatory bowel disease, psoriasis, psoriatic arthritis, rheumatoid arthritis (RA), Sjögren's syndrome and systemic lupus erythematosus (SLE); wherein said inflammatory disease is selected from the group consisting of allergy, amyotrophic lateral sclerosis (ALS), asthma, chronic inflammatory disorder, atopic dermatitis, coronary atherosclerosis, interstitial cystitis, diabetes mellitus type 1 (IDDM), idiopathic thrombocytopenic purpura, multiple sclerosis and chronic pancreatitis; and wherein said neurodegenerative disease is selected from the group consisting of autism spectrum disorders (ASD), chronic fatigue syndrome, chronic prostatitis, fibromyalgia, vitiligo and Parkinson's Disease.

18. The kit of claim 8 wherein said disease is selected from the group consisting of rheumatoid arthritis, psoriasis, autism spectrum disorders (ASD), SLE and mastocytosis.

19. -27. (canceled)
